(19) United States
(12) Patent Application Publication
(10) Pub. No.: US 2020/0224165 A1
(43) Pub. Date: Jul. 16, 2020

(54) METHODS AND COMPOSITIONS FOR REJUVENATION

(71) Applicants: Senlin LI, (US); Robert A CLARK, San Antonio, TX (US); Cang CHEN, San Antonio, TX (US); Michael J GUDERYON, San Antonio, TX (US)

(72) Inventors: Senlin LI, (US); Robert A CLARK, San Antonio, TX (US); Cang CHEN, San Antonio, TX (US); Michael J GUDERYON, San Antonio, TX (US)

(21) Appl. No.: 16/319,517

(22) PCT Filed: Jul. 7, 2017
(86) PCT No.: PCT/US2017/041088

§ 371 Date: Jan. 22, 2019

Related U.S. Application Data
(60) Provisional application No. 62/365,492, filed on Jul. 22, 2016.

(51) Int. Cl. C12N 5/0789

(52) U.S. Cl. CPC C12N 5/0647; C12N 2501/22

(57) ABSTRACT

Certain embodiments are directed to compositions and methods for non-cytotoxic hematopoietic stem cell transplantation.

### PRIORITY CLAIM

This Application claims priority to U.S. Provisional Patent Application 62/365,492 filed Jul. 22, 2016, which is incorporated herein by reference in its entirety.

### STATEMENT REGARDING FEDERALLY FUNDED RESEARCH

Certain embodiments of this invention were made with government support under I01BX007080 awarded by the Veterans Administration. The government has certain rights in the invention.

### BACKGROUND

The number of elderly in our population is increasing. Breakthroughs in biomedical research aiming to increase healthspan and lifespan will dramatically improve the quality of life for these elderly individuals and create economic and other benefits to society as a whole. The field of aging research has now moved into developing interventions that enhance healthspan and lifespan. Novel pharmacologic, biologic, and genetic interventions that have potential to extend lifespan and delay cancers, dementias, and other age-related diseases are being explored. However, there are many caveats and limitations. For example, rapamycin has been shown to extend lifespan as well as healthspan in mice, but the mechanism accounting for these effects remains elusive and a growing list of side effects raises some doubts as to whether this drug will be beneficial in man.

There is a need for additional compositions and methods for increasing healthspan and lifespan.

### SUMMARY

Certain embodiments are directed to an intervention that extends healthspan and lifespan by blood cell rejuvenation. Blood cells, all derived from hematopoietic stem cells (HSCs), are responsible for constant maintenance and immune protection of all tissues in the body. Age-related declines in HSCs and their progeny blood cells contribute to poor tissue oxygenation, impaired hemostasis, and decreased immune protection, as well as increased chronic inflammation, immune activation, and tumorigenesis in the elderly, leading to both morbidity and mortality. Blood cell rejuvenation can be achieved by a new form of hematopoietic stem cell transplantation (HSCT), which can be free of side effects associated with conventional HSCT, which is associated with irradiation or cytotoxic chemotherapeutic agents used as a conditioning regimen (e.g., infection, immune reactions, bleeding). The rejuvenation of blood cells can lead to healthspan and lifespan extension.

Certain embodiments of the invention provide methods for non-cytotoxic HSCT. Non-cytotoxic HSCT includes methods that do not use chemotherapy or irradiation to condition the subject prior to administration of transplant or replacement cells. In certain aspects, the HSCT methods described herein include administering a stem cell mobilization agent to stimulate migration of target stem cells out of a stem cell niche, followed by the administration of exogenous (e.g., transplant or replacement) stem cells that subsequently migrate to the appropriate stem cell niche. As used herein exogenous stem cells refers to stem cells other than those stem cells occupying the stem cell niche at the time of mobilization. Thus, exogenous stem cells include stem cells previously isolated from the same patient and returned to that same patient at a later time. In certain aspects this mobilization and transplantation cycle is performed for a number of cycles. In a further aspect the mobilization/transplantation cycle is performed at least four times.

As used herein, a “stem cell niche” is a tissue microenvironment where stem cells are found, and the microenvironment interacts with stem cells to regulate stem cell fate. The word ‘niche’ can be in reference to the in vivo stem cell microenvironment. In the body, stem cell niches maintain stem cells in a quiescent state, but after activation, the surrounding microenvironment actively signals to stem cells to promote either self-renewal or differentiation to form new cells or tissues. Several factors contribute to the characteristics within a particular niche: (i) cell-cell interactions between stem cells, and between stem cells and neighboring cells; (ii) interactions between stem cells and adhesion molecules, extracellular matrix components, growth factors, and cytokines; and (iii) the physiochemical nature of the microenvironment including oxygen tension, pH, ionic strength (e.g., Ca²⁺ concentration) and presence of various metabolites. The mobilization of the target stem cells (the movement from or evacuation of a niche) increases the probability that a transplant or replacement stem cell will occupy the stem cell niche.

The “target stem cell” is defined as an endogenous stem cell that is mobilized, collected, and/or depleted from a subject. A “transplant or replacement stem cell” is a stem cell that is being introduced to a subject. The transplant or replacement stem cell can be a therapeutic stem cell in that it has been conditioned or otherwise modified to be therapeutic to the subject.

Certain embodiments are directed to methods of non-cytotoxic stem cell transplant or replacement comprising: (a) administering at least one stem cell mobilization agent to a subject, wherein a target stem cell population migrates from a host stem cell niche into the subject's circulating blood compartment; (b) removing the mobilized target stem cells from the subject (e.g., apheresis); (c) administering transplant or replacement stem cells to the subject, wherein the transplant or replacement stem cells migrate to and occupy the host stem cell niche; and (d) repeating steps (a)-(c) 2, 3, 4, 5, 6, 7, 8, 9, or more times. In certain aspects the transplant or replacement stem cells are therapeutic or rejuvenated stem cells. In further aspects the therapeutic stem cells are isolated target stem cells that have been manipulated in vitro. In certain aspects the transplant, replacement, and/or therapeutic stem cells are isolated from the subject to be treated. In other aspects the transplant, replacement, and/or therapeutic stem cells are isolated from a heterologous source, i.e., a source or donor that is not the subject to be treated. The term “isolated” refers to a cell, a nucleic acid, or a polypeptide that is substantially free of heterologous cells or cellular material, bacterial material, viral material, and/or culture medium of their source of origin; or chemical precursors or other chemicals when chemically synthesized. A donor can be an autologous, allogeneic, or xenogeneic (a non-genetically identical donor of another species) donor. In certain aspects the therapeutic stem cells are genetically engineered. In certain aspects the transplant or replacement stem cells are from an autologous donor. In a further aspect the transplant or replacement stem cells are from an allogeneic donor. In a still further aspect the transplant or replacement cells are from a xenogeneic donor. In certain aspects the target stem cell is a hematopoietic stem cell. In certain aspects the transplant or replacement stem cell is a hematopoietic stem cell or a hematopoietic stem cell precursor cell.

In certain aspects a mobilization agent can be selected from interleukin-17 (IL-17), AMD3100, granulocyte-colony stimulating factor (G-CSF), Ancestim, anti-sense VLA-4 receptor (e.g., ATL1102, (Antisense Therapeutics Limited)), POL6326, BKT 140, NOX-A12, Natalizumab, sphigosine-1-phosphate (S1P) agonists, hypoxia-inducible factor, and/or other agents known to mobilize stem cells. In certain aspects the mobilization agent is granulocyte-colony stimulating factor. In certain aspects a mobilization agent includes AMD3100. In a further embodiment the subject is administer both G-CSF and AMD3100. In a further aspect the mobilization agent can be administered prior to or during administration of the transplant or replacement stem cells to the subject.

In certain aspects the isolated target stem cells are manipulated by genetically modifying and/or in vitro conditioning the isolated cells from the subject.

The terms “individual,” “host,” “subject,” and “patient” are used interchangeably to refer to an animal that is the object of treatment, observation and/or experiment. “Animal” includes vertebrates, such as mammals. “Mammal” includes, without limitation, mice, rats, rabbits, guinea pigs, dogs, cats, sheep, goats, cows, horses, primates, such as monkeys, chimpanzees, and apes, and humans. In certain embodiments the subject is a human subject.

The terms “ameliorating,” “treating,” “treatment,” “therapeutic,” or “therapy” do not necessarily mean total cure or abolition of the disease or condition. Any alleviation of any undesired signs or symptoms of a disease or condition, to any extent, can be considered amelioration, and in some respects a treatment and/or therapy.

As used herein, the term “progenitor cells” refers to cells that, in response to certain stimuli, can form differentiated cells, such as hematopoietic or myeloid cells. As used herein, “stem” cells are less differentiated forms of progenitor cells. Typically, such cells are often positive for CD34 in humans.

The term “providing” is used according to its ordinary meaning “to supply or furnish for use.” In some embodiments, a protein is provided by administering the protein, while in other embodiments, the protein is effectively provided by administering a nucleic acid that encodes the protein or a cell that synthesizes the protein.

Other embodiments of the invention are discussed throughout this application. Any embodiment discussed with respect to one aspect of the invention applies to other aspects of the invention as well and vice versa. Each embodiment described herein is understood to be an embodiment of the invention that is applicable to all aspects of the invention. It is contemplated that any embodiment discussed herein can be implemented with respect to any method or composition of the invention, and vice versa. Furthermore, compositions and kits of the invention can be used to achieve methods of the invention.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1A-1B. (A) Effect of G-CSF aided young HSC transplantation (HSCT) on food intake of aged GFP+ mice. Mice were group-housed in standard mouse cages with ad libitum access to food and water. Food intake was measured monthly and determined daily at same time for 5 days. Each point represents mean±SEM of daily food intake for individual from surviving animals. * P<0.05, ** P<0.01 vs. controls. (B) Effect of G-CSF aided young HSC transplantation (HSCT) on body weight of aged GFP+ mice. Each point represents mean±SEM from surviving animals. * P<0.05, ** P<0.01 vs. controls.

FIG. 2A-2B. Effect of G-CSF-aided young HSC transplantation on horizontal and vertical locomotor activities of aged mice. The horizontal (A) and vertical (B) locomotor activities of G-CSF and control mice were tested monthly by the Photobeam Activity System (San Diego Instruments, San Diego, Calif.) after young HSC transplantation. The locomotor activities were recorded for 60 min and assessed at 10-min time intervals. Each point represents mean of 6×10 min recorded from surviving animals per group. Error bars indicate ±SEM. * P<0.05 vs. control group.

FIG. 3A-3B. Effect of G-CSF aided young HSC transplantation on lifespan of aged GFP mice. 22-month old GFP mice were 8 times transplanted with 10-week old HSCs (1.0×10⁶ enriched lineage-negative cells per animal) after G-CSF and AMD3100 preconditioning, while same aged control mice were received vehicle without HSCs transplantation. The survival curves show longer lifespan in young HSC-transplanted aged mice when compared to control mice (P<0.0001).

FIG. 4A-4B. Effect of G-CSF aided young HSC transplantation on hematopoiesis in aged GFP mice. (A) Data showing T cells (CD3ε+) were significantly higher in PB of young HSC transplanted aged mice compared to same aged control mice, but no difference were found in B cells (B220+) and myeloid cell populations. (B) There were no statistically significant differences in the percentage of LMPPs, ST-HSCs and LT-HSCs among LSK BM cells between young HSC transplanted aged mice and control mice. Each bar represents mean±SEM from five animals. ** P<0.01.

### DESCRIPTION

The field of aging research has now moved into developing interventions that enhance both healthspan and lifespan in experimental animals. However, these interventions have many unsolved caveats and limitations. Take the front-runner rapamycin as an example. This compound has been tested in mice and shown to extend mean and maximum lifespans, as well as healthspan, but a growing list of side effects raises question as to whether this drug will ultimately be beneficial to humankind. Stem cell-based regenerative medicine is believed to be a key element to retard aging. However, effective therapeutic delivery/transplantation of stem cells is challenging. Hematopoietic stem cells can be transplanted effectively, but the required preconditioning is toxic and risky. Therefore, conventional HSCT is not suitable for anti-aging or regenerative interventions. Embodiments described herein provide suitable methods and compositions for anti-aging or regenerative treatments.

Interventions described herein may be applied in various scenarios: (1) PBSCs can be collected from young adults by apheresis after s.c. injections of G-CSF and/or other HSC mobilizers and then cryopreserved. This process can be repeated multiple times so sufficiently large numbers of cells can be stored. Once these individuals become aged, their old-phenotype blood cells can be replaced and repopulated by the autologous PBSCs previously collected and stored (i.e., young phenotype cells). (2) Alternatively, multiple batches of PBSCs could be collected from the elderly. In certain aspects the cells can be, but need not be, cryopreserved. The HSCs from these PBSCs can be rejuvenated in vitro by genetic (e.g., over-expression of Sirt3) or by pharmacologic (e.g., treatment with cdc42 inhibitors) manipulation (conditioned) and then transplanted back into the same individuals using the methods described herein. (3) Another potential source of therapeutic or treatment

HSCs can be autologous reprogrammed pluripotent stem cells (e.g. iPS cells).

Hematopoietic stem cell transplantation (HSCT) is used in the treatment of a variety of hematological, autoimmune, and malignant diseases. HSCT is the transplantation of blood stem cells derived from the bone marrow (in this case known as bone marrow (BM) transplantation), blood (such as peripheral blood and umbilical cord blood), or amniotic fluid. Currently, patients endure a harsh conditioning regimen prior to HSCT known as myeloablation to eradicate the disease and hematopoietic stem cells (HSCs). “Myeloablation” refers to the severe or complete depletion of HSCs by the administration of chemotherapy and/or radiation therapy prior to HCST. This treatment severely impacts the myeloproliferative function of the hematopoietic system. Myeloablation techniques for allogeneic transplants (the transplantation of cells, tissues, or organs to a recipient from a genetically non-identical donor of the same species) can include a combination of cyclophosphamide with busulfan or total body irradiation (TBI). Autologous transplants (the transplantation of cells, tissues, or organs to a recipient from a genetically identical donor, e.g., the subject is both the recipient and the donor) may also use similar regimens. Various chemotherapy and/or radiation combinations can be used depending on the disease.

The indiscriminate destruction of HSCs can lead to a reduction in normal blood cell counts, such as lymphocytes, neutrophils, and platelets. Such a decrease in white blood cell counts also results in a loss of immune system function and increases the risk of acquiring opportunistic infections. Neutropenia resulting from chemotherapy and/or radiation therapy may occur within a few days following treatments. The subject remains vulnerable to infection until the neutrophil counts recover to within a normal range. If the reduced leukocyte count (leukopenia), neutrophil count (neutropenia), granulocyte count (granulocytopenia), and/or platelet count (thromboocytopenia) become sufficiently serious, therapy must be interrupted to allow for recovery of the white blood cell and/or platelet counts.

There are “non-myeloablative” conditioning regimens being tested using lower dose chemotherapy and/or radiation therapy that do not eradicate all of the hematopoietic cells, but the subjects still suffer similar side effects, just to a lesser degree. Notably, the treatment of non-malignant diseases by autologous HSCT does not require cytotoxic conditioning regimens. For example, current experimental non-myeloablative conditioning regimens include antibody-based (Czechowicz et al. Science. 2007, 318(5854):1296-99; Xue et al. Blood. 2010, 116:5419-22), type I interferon-mediated (Sato et al. Blood. 2013, 121(16):3267-73), and G-CSF-modulated pre-transplant conditioning (Mardiney and Malech, Blood. 1996, 87(10):4049-56; Barese et al. Stem Cells. 2007, 25(6)1578-85). However, the antibody-mediated conditioning regimen (Czechowicz et al.) works only in immune-deficient subjects, not for HSCT recipients that are immune-competent. Type I interferon-mediated and G-CSF-modulated pre-transplant conditioning regimens still require irradiation or chemotherapy, but at reduced (non-myeloablative) doses. AMD3100 was tried without irradiation and chemotherapy and shown not to be sufficiently effective. Embodiments of methods described herein provide an effective “non-cytotoxic” regimen (i.e., a regimen with little to no cytotoxicity) so that the side effects of irradiation and chemotherapy are avoided.

### I. STEM CELL TRANSPLANTATION OR REPLACEMENT

Stem cells are undifferentiated cells that can differentiate into specialized cells and can divide (through mitosis) to produce more stem cells. In mammals, there are two broad types of stem cells: (i) embryonic stem cells, which are isolated from the inner cell mass of blastocysts, and (ii) adult stem cells, which are found in various tissues. In adult organisms, stem cells and progenitor cells act as a repair system for the body, replenishing adult tissues. Usual sources of adult stem cells in humans include bone marrow (BM), adipose tissue (fat cells), and blood. Harvesting stem cells from blood can be done through apheresis, wherein blood is drawn from a donor (similar to a blood donation), and passed through a machine that extracts stem cells and returns other portions of the blood to the donor. Another source of stem cells is umbilical cord blood.

Adult stem cells are frequently used in medical therapies, for example in bone marrow transplantation. Stem cells can now be grown, manipulated, and/or transformed (differentiated) into specialized cell types with characteristics consistent with cells of various tissues such as muscles or nerves. Embryonic cell lines and autologous embryonic stem cells generated through therapeutic cloning have also been proposed as promising candidates for therapies.

Autologous harvesting of stem cells is one of the least risky methods of harvesting. By definition, autologous cells are obtained from one's own body, just as one may bank his or her own blood for elective surgical procedures, one may also bank stem cells. Autologous stem cell transplantation is a medical procedure in which stem cells are removed, stored, and/or reintroduced into the same person. These stored cells can then be the source for transplant or replacement stem cells in the methods described herein.

Stem cell transplants are most frequently performed with hematopoietic stem cells (HSCs). Autologous HSCT comprises the extraction of HSCs from the subject and/or freezing of the harvested HSCs. After conditioning or genetic engineering of cells isolated from the subject, the subject's HSCs are transplanted into the subject. Allogeneic HSCT involves HSC obtained from an allogeneic HSC donor. Typically the allogeneic donor has a human leukocyte antigen (HLA) type that matches the subject.

Embodiments of the non-cytotoxic methods described herein comprise mobilizing a target stem cell population (inducing the movement of the stem cells to the blood or other body fluid); removing, isolating, and/or selecting a the target stem cell population from the stem cell-enriched body fluid; administering a transplant or replacement stem cell population to a subject, wherein the transplant or replacement stem cell population localizes in the niche for the target stem cell population. In certain aspects the steps of the method are repeated a number of times. Multiple rounds of transplantation can lead to an increasing representation of the transplant or replacement stem cell population in the subject.

In certain aspects hematopoietic stem cells are mobilized from their niche in the bone marrow and replaced with a therapeutic stem cell. Hematopoietic stem cells (HSCs) are bone marrow cells with the capacity to reconstitute the entire hematopoietic system. Hematopoietic stem cells are identified by their small size, lack of lineage (lin⁻) markers, low staining with vital dyes such as rhodamine (rhodamine-DULL, also called rholo), and presence of various antigenic markers on their surface. A number of the HSC markers belong to the cluster of differentiation series, like: CD34, CD38, CD90, CD133, CD105, CD45, and also c-kit (stem cell factor receptor). The hematopoietic stem cells are negative for markers used to detect lineage commitment, and are, thus, called Lin-minus (Lin-). Blood-lineage markers include but are not limited to CD13 and CD33 for myeloid, CD71 for erythroid, CD19 for B lymphocytes, CD61 for megakaryocytes for humans; and B220 (murine CD45) for B lymphocytes, Mac-1 (CD11b/CD18) for monocytes, Gr-1 for granulocytes, Ter119 for erythroid cells, Il7Ra, CD3, CD4, CD5, CD8 for T lymphocytes, etc. in mice. Antibodies can be used to deplete the lin+ cells.

Stem cells can include a number of different cell types from a number of tissue sources. The term “induced pluripotent stem cell” (iPS cell) refers to pluripotent cells derived from mesenchymal cells (e.g., fibroblasts and liver cells) through the over-expression of one or more transcription factors. In certain aspects iPS cells are derived from fibroblasts by the over-expression of Oct4, Sox2, c-Myc, and Klf4 (Takahashi et al. Cell, 126:663-76, 2006 for example). As used herein, “cells derived from an iPS cell” refers to cells that are either pluripotent or terminally differentiated as a result of the in vitro culturing or in vivo transplantation of iPS cells.

Neural stem cells are a subset of pluripotent cells that have partially differentiated along a neural cell pathway and express some neural markers, including for example nestin. Neural stem cells may differentiate into neurons or glial cells (e.g., astrocytes and oligodendrocytes).

A population of cells can be depleted of cells expressing certain surface markers using a selection process that removes at least some of the cells expressing various cell surface markers. This selection process may be done by any appropriate method that preserves the viability of the cells that do not express the selection marker, including for example, fluorescence-activated cell sorting (FACS) or magnetically-activated cell sorting (MACS). Preferably, depleted populations contain less than 10%, less than 5%, less than 2.5%, less than 1%, or less than 0.1% of cells expressing the selection marker.

Hematopoietic stem cells reside in specific niches in the bone marrow (BM) that control survival, proliferation, self-renewal, or differentiation. In normal individuals, the continuous trafficking of HSCs between the BM and blood compartments likely fills empty or damaged niches and contributes to the maintenance of normal hematopoiesis (Wright et al. Science. 2001, 294:1933-36; Abkowitz et al. Blood. 2003, 102:1249-53). It has been known for many years that egress of HSCs can be enhanced by multiple agonists known as “stem cell mobilization agents.” The hematopoietic cytokine granulocyte-colony stimulating factor (G-CSF), a glycoprotein that stimulates the bone marrow to produce granulocytes and stem cells and release them into the bloodstream, is widely used clinically to elicit HSC mobilization for BM transplantation (Lapidot and Petit. Exp. Hematol. 2002, 30:973-81; Papayannopoulou, T. Blood. 2004, 103:1580-85). Functionally, it is a cytokine and hormone, a type of colony-stimulating factor, and is produced by a number of different tissues. In addition, AMD3100 has been shown to increase the percentage of persons that respond to the therapy and functions by antagonizing CXCR4, a chemokine receptor important for HSC homing to the BM. In certain aspects a subject is administered an agent that induces movement of a stem cell from the niche and an agent that inhibits the homing of a stem cell to the niche.

The dosages and dosage regimen in which the mobilization agents are administered will vary according to the dosage form, mode of administration, the condition being treated and particulars of the patient being treated. Accordingly, optimal therapeutic concentrations will be best determined empirically at the time and place through routine experimentation.

Certain mobilization agent(s) may be administered parenterally in the form of solutions or suspensions for intravenous or intramuscular perfusions or injections. In that case, the mobilization agent(s) are generally administered at the rate of about 10 μg to 10 mg per day per kg of body weight. Methods of administration include using solutions or suspensions containing approximately from 0.01 mg to 1 mg of active substance per ml. In certain aspects the mobilization agent(s) are administered at the rate of about 10, 20, 30, 40, 50, 60, 70, 80, 90, or 100 μg to 1, 2, 3, 4, 5, 6, 7, 8, 9, or 10 mg per day per kg of body weight.

Certain mobilization agents may be administered enterally. Orally, the mobilization agent(s) can be administered at the rate of 100 μg to 100 mg per day per kg of body weight. In certain aspects the mobilization agent(s) can be administered at the rate of about 100, 150, 200, 250, 300, 350, 400, 450, or 500 μg to about 1, 5, 10, 25, 50, 75, 100 mg per day per kg of body weight. The required dose can be administered in one or more portions. For oral administration, suitable forms are, for example, tablets, gel, aerosols, pills, dragees, syrups, suspensions, emulsions, solutions, powders and granules.

The agent(s) and/or pharmaceutical compositions disclosed herein can be administered according to various routes, typically by injection, such as local or systemic injection(s). However, other administration routes can be used as well, such as intramuscular, intravenous, intradermic, subcutaneous, etc. Furthermore, repeated injections can be performed, if needed.

For in vivo administration, active agent(s) can be added to, for example, a pharmaceutically acceptable carrier, e.g., saline and buffered saline, and administered by any of several means known in the art. Examples of administration include parenteral administration, e.g., by intravenous injection including regional perfusion through a blood vessel supplying the tissues(s) or organ(s) having the target cell(s), or by inhalation of an aerosol, subcutaneous or intramuscular injection, topical administration such as to skin wounds and lesions, direct transfection into, e.g., bone marrow cells prepared for transplantation and subsequent transplantation into the subject, and direct transfection into an organ that is subsequently transplanted into the subject. Further administration methods include oral administration, particularly when the active agent is encapsulated.

In contrast to difficult bone marrow transplants, HSCs can be easily collected from the peripheral blood and this method provides a bigger graft, does not require that the donor be subjected to general anesthesia to collect the graft, results in a shorter time to engraftment, and may provide for a lower long-term relapse rate. In order to harvest HSCs from the circulating peripheral blood, subjects are administered one or more mobilization agents that induce cells to leave their niche and circulate in the blood. The subjects then undergo apheresis to enrich and collect the HSCs and then return the HSC-depleted blood to the subjects.

The compositions can be administered using conventional modes of delivery including, but not limited to, intravenous, intraperitoneal, oral, subcutaneous, intraarterial, and by perfusion through a regional catheter. When administering the compositions by injection, the administration may be by continuous infusion or by single or multiple boluses. For parenteral administration, the stem cell mobilization agents may be administered in a pyrogen-free, parenterally acceptable aqueous solution comprising the desired stem cell mobilization agents in a pharmaceutically acceptable vehicle. A particularly suitable vehicle for parenteral injection is sterile distilled water in which one or more stem cell mobilization agents are formulated as a sterile, isotonic solution, properly preserved.

The methods described herein provide gentle and low-risk, but high-level, replacement of endogenous stem cells with either genetically engineered or pharmacologically rejuvenated (i.e., conditioned) HSCs or the combination.

Cells may be cultured and (i) expanded to increase the population of stem cells, (ii) genetically engineered and/or (iii) otherwise conditioned, prior to reintroduction of such cells into a patient. These stem cells or precursor cells may be used for ex vivo gene therapy, whereby the cells may be transformed (i.e., genetically engineered) in vitro prior to reintroduction of the transformed cells into the patient. In gene therapy, using conventional recombinant DNA techniques, a selected nucleic acid, such as a gene, may be isolated, placed into a vector, such as a viral vector, and the vector transfected into a stem cell, to transform the cell, and the cell may in turn express the product encoded by the gene. The cell then may then be introduced into a patient (Wilson et al. PNAS. 1998, 85:3014-18). However, there have been problems with efficient hematopoietic stem cell transfection (Miller. Blood. 1990, 76:271-78). A transformed cell can be engineered to express and/or secrete a therapeutic protein such as a growth factor, cytokine, monoclonal antibody (positive modulator of another protein or cell or a negative modulator of another protein or cell), ligand, enzyme, receptor, etc.

Ex vivo administration of active agents can be done by any standard method that would maintain viability of the cells, such as by adding it to culture medium (appropriate for the target cells) and adding this medium directly to the cells. As is known in the art, any medium used in this method can be aqueous and non-toxic so as not to render the cells non-viable. In addition, it can contain standard nutrients for maintaining viability of cells, if desired.

### II. REJUVENATION METHODS

Currently there are more than 39 million Americans aged 65 or older. Breakthroughs in biomedical research aiming to increase healthspan and lifespan will create economic benefit and dramatically improve the quality of life for these elderly individuals, as well as to society as a whole. The field of aging research has now moved into developing interventions that enhance healthspan and lifespan in experimental animals. Novel pharmacologic, biological, and genetic interventions have potential to extend lifespan, delay cancers, dementias, and possibly other age-related diseases. However, these interventions have many caveats and limitations. For example, rapamycin has been shown to extend lifespans as well as healthspan in mice, but the mechanism accounting for these effects remains elusive and a growing list of side effects raises some doubts as to whether this drug will be beneficial in man.

Methods described herein can be used to extend healthspan and lifespan by rejuvenation of blood cells. Blood cells, all derived from hematopoietic stem cells (HSCs), are responsible for constant maintenance and immune protection of every cell type of the body. Age-related declines in HSCs and their progeny blood cells contribute to poor tissue oxygenation, impaired hemostasis, and decreased immune protection, as well as increased chronic inflammation and tumorigenesis (two common health problems in the elderly), which may eventually lead to ailments and deaths. The rejuvenation of blood cells can be achieved using hematopoietic stem cell transplantation (HSCT) as described herein.

The ability to replace HSCs using the methods described herein is the basis for the development of a mobilization-based conditioning regimen. Data in inbred mouse models showed ˜75% transplantation efficiency after multiple repetitions of this procedure. These methods can be used to introduce younger or rejuvenated stem cells into a subject.

The rejuvenation of blood cells can lead to healthspan and lifespan extension. A mouse model can be used that replaces old HSCs with young ones. For example, rejuvenation of blood cells by replacement for healthspan extension can be demonstrated using 20 female and 20 male C57BL/6 mice at 19 months of age that are transplanted with either age-matched old HSCs (control) or young HSCs (derived from 10-week old) by the methods described herein. Health assessments are done monthly by measurement of motor and cognitive functions using 50-hour home cage activity, stride length, grip strength, Y-maze, and novel object tests. Transplantation efficiency of 80-90% and blood cell rejuvenation is verified by characterization of blood cells at 26 and 32 months of age. In a second part of the study 36 female and 44 male C57BL/6 mice at 19 months of age are transplanted as above. Animal survival is monitored and recorded. End of life pathology is performed.

In humans, this intervention may be applied (1) where PBSCs are collected from young adults by apheresis after s.c. injections of G-CSF and/or other HSC mobilizer(s) (e.g., G-CSF) (NEUPOGEN® and AMD3100 (MOZOBIL™)) and then cryopreserved, as currently practiced in clinic. This process is repeated multiple times (twice a year, for instance) so sufficiently large numbers of cells are stored. Once these individuals have aged, their old-phenotype blood cells would be replaced and repopulated by the young PBSCs that were obtained and stored when they were young. The replacement could reach ˜90% through repeated mobilization conditioning-based transplantations of the young PBSCs. The technology and reagents are readily applicable in today's clinic. (2) Alternatively, multiple batches of PBSCs could be collected from the elderly and cryopreserved. The HSCs from these PBSCs could be rejuvenated in vitro by genetic (over-expression of Sirt3) or by pharmacologic manipulation (treatment with cdc42 inhibitors) and transplanted back into the same individuals using the conditioning regimen and transplant method described. The HSCs can be treated ex vivo in culture with cdc42 inhibitor (CASIN) for 8-16 hours and then transplanted back to the same subjects (Florian et al., 2012) or genetically engineered to over-express SirT3 (Brown et al., 2013). (3) Another potential source of youthful HSCs would be autologous reprogrammed pluripotent stem cells (such as iPS cells). Skin or blood cells can be collected from elderly patients and converted to induced pluripotent stem cells (iPS cells). The iPS cells are differentiated into HSCs, which are transplanted into the same subject (Hanna et al., 2007). The transplantation is done repeatedly to achieve sufficient replacement of HSCs.

### III. EXAMPLES

The following examples, as well as the figures, are included to demonstrate preferred embodiments of the invention. It should be appreciated by those of skill in the art that the techniques disclosed in the examples or figures represent techniques discovered by the inventors to function well in the practice of the invention, and thus can be considered to constitute preferred modes for its practice. However, those of skill in the art should, in light of the present disclosure, appreciate that many changes can be made in the specific embodiments that are disclosed and still obtain a like or similar result without departing from the spirit and scope of the invention.

### Example 1

### Non-Cytotoxic HSCT

Methods and compositions described herein are to extend healthspan and lifespan by blood cell rejuvenation. Blood cells, all derived from hematopoietic stem cells (HSCs), are responsible for constant maintenance and immune protection of all tissues in the body. Age-related declines in HSCs and their progeny blood cells contribute to poor tissue oxygenation, impaired hemostasis, and decreased immune protection, as well as increased chronic inflammation, immune activation, and tumorigenesis in the elderly, leading to both morbidity and mortality.

HSCs reside in specialized niches in the bone marrow, wherein the majority of HSCs reside, but some (˜2%) leave their niches and travel in the blood. The egress of HSCs from bone marrow creates empty niches that are ready to host new incoming HSCs. The egress of HSCs can be dramatically increased by mobilization using G-CSF, alone or in combination with other agents. This leads to increased numbers of HSCs in the peripheral blood, along with empty niches in the bone marrow. The former comprises the rationale for collection of HSCs from peripheral blood (PBSCs) in the clinic, whereas the latter phenomenon is the basis for the development of a novel mobilization-based conditioning regimen. When the empty niches reach a peak in number, conventional i.v. injection/infusion of a large number of syngeneic (mouse) or autologous (human) HSCs are performed in order to provide them with a competitive advantage over endogenous circulating HSCs for occupation of the available niches in the bone marrow. Indeed, by vigorous optimization, preliminary studies in inbred mouse models repeatedly showed ˜90% transplantation efficiency after multiple repetitions of this procedure. The rejuvenation of blood cells can lead to healthspan and lifespan extension. A mouse model is used to demonstrate this principle by replacing old HSCs with young ones.

One aim is to test whether rejuvenation of blood cells by replacement will lead to healthspan extension. Forty female C57BL/6 mice at 19 months of age per group will be used as recipients. Three groups undergo G-CSF mobilization conditioning, followed by syngeneic transplantation with young or old HSCs in two groups, while the third will receive vehicle only (no cells). A fourth group experiences all the procedures, but with vehicle in place of both G-CSF and HSCs. A fifth group is maintained intact as transplantation procedure controls.

Motor function, is used as an indicator of health, is assessed monthly by tests of locomotion, strength, balance, and endurance. Transplantation efficiency of 80-90% and blood/immune cell rejuvenation will be verified and characterized at 26 and 32 months of age. The mice are sacrificed for gross pathology and histopathological assessment, including lymphoma incidence and severity.

A second aim of the studies described herein is to test whether rejuvenation of blood cells by replacement will lead to lifespan extension. The same design as above is used, and survival is monitored. Carcasses will be preserved and end-of-life pathology performed.

This exploratory/developmental study provides proof-of-concept data to support further development of cell-based anti-aging interventions. In humans, this intervention may be applied in a couple of scenarios: (1) PBSCs would be collected from young adults by apheresis after s.c. injections of G-CSF and/or other HSC mobilizers and then cryopreserved, as currently practiced in clinic. This process could be repeated multiple times (twice a year, for instance) so sufficiently large numbers of cells could be stored. Once these individuals become aged, their old-phenotype blood cells would be replaced and repopulated by the autologous PBSCs that were obtained and stored when they were young. The replacement could reach a desired level (up to ˜90%) through repeated mobilization conditioning-based transplantations of the young PBSCs. The technology and reagents are readily applicable in today's clinic. (2) Alternatively, multiple batches of PBSCs could be collected from the elderly and cryopreserved. The HSCs from these PBSCs could be rejuvenated in vitro by genetic (over-expression of Sirt3) or by pharmacologic (treatment with cdc42 inhibitors) manipulation and then transplanted back into the same individuals using our novel conditioning regimen and transplant method. (3) Another potential source of youthful HSCs would be autologous reprogrammed pluripotent stem cells (such as iPS cells).

In a preliminary study, thirty two of 22-month old, body weight matched female GFP transgenic mice were divided into two groups (16 animals per group). Group 1 (G-CSF) were given G-CSF combined with AMD3100 and then transplanted with young HSCs derived from 10-week old C57BL/6J mice according to the novel HSCT procedure. Group 2 (Control) received vehicle without HSCs transplantation. The same HSCT procedures were applied 8 times at one week intervals between each procedure. The efficacy of HSC transplantation was assessed by determination of percentage of GFP+ cells in peripheral blood by flow cytometry (BD FACSCalibur System, BD Bioscience, San Jose, Calif.) and fluorescent microscope examination (Nikon Eclipse TE2000-U, Nikon Instruments, Melville, N.Y.). Table 1 shows that up to 90% accumulated transplantation efficiency was reached after 8 HSCT procedure applied.

TABLE 1
G-CSF Mobilization-aided HSC transplantation

| | Replacement | Replacement | Replacement |
|---|---|---|---|
| | result (%) | result (%) | result (%) |
| Transplantation | Model-based | from young | from young |
| repeats (n) | calculation | to young | to old |
| 1 | 20.0 | 22.1 ± 7.78 | 20.83 ± 1.10 |
| 2 | 36.00 | 31.4 ± 1.37 | 30.23 ± 3.82 |
| 3 | 48.80 | 39.1 ± 6.50 | 43.40 ± 3.14 |
| 4 | 59.04 | | 50.25 ± 1.05 |
| 5 | 67.23 | | 60.43 ± 1.44 |
| 6 | 73.79 | 63.2 ± 4.29 | 72.99 ± 1.28 |
| 7 | 79.03 | 74.4 ± 6.63 | 82.28 ± 5.50 |
| 8 | 83.22 | 85.2 ± 5.10 | 90.43 ± 1.17 |
| 9 | 86.58 | 91.2 ± 3.62 | |

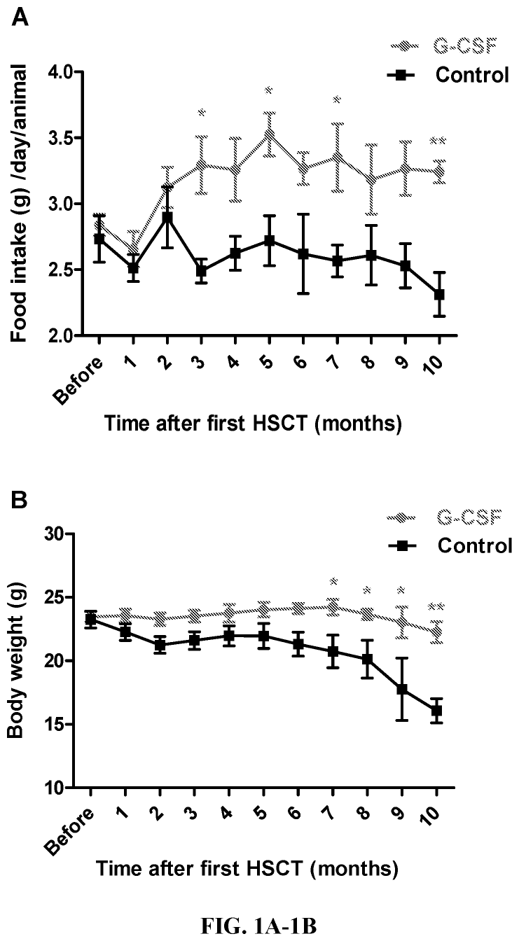

Effect of G-CSF aided young HSC transplantation on food intake and body weight of aged GFP mice—Food intake and body weight were measured monthly until animal deceased. Data shows that the young HSC transplanted mice consumed more food and their body weight were higher than the control mice during the surviving time (FIG. 1A and FIG. 1B).

Effect of G-CSF aided young HSC transplantation (HSCT) on horizontal and vertical locomotor activities of aged mice—In preliminary studies, the locomotor activity was designed as one of the indicator for assessment of healthspan. The horizontal and vertical locomotor activities were tested monthly by the Photobeam Activity System (San Diego Instruments, San Diego, Calif.) before and after young HSCT procedure until animal deceased.

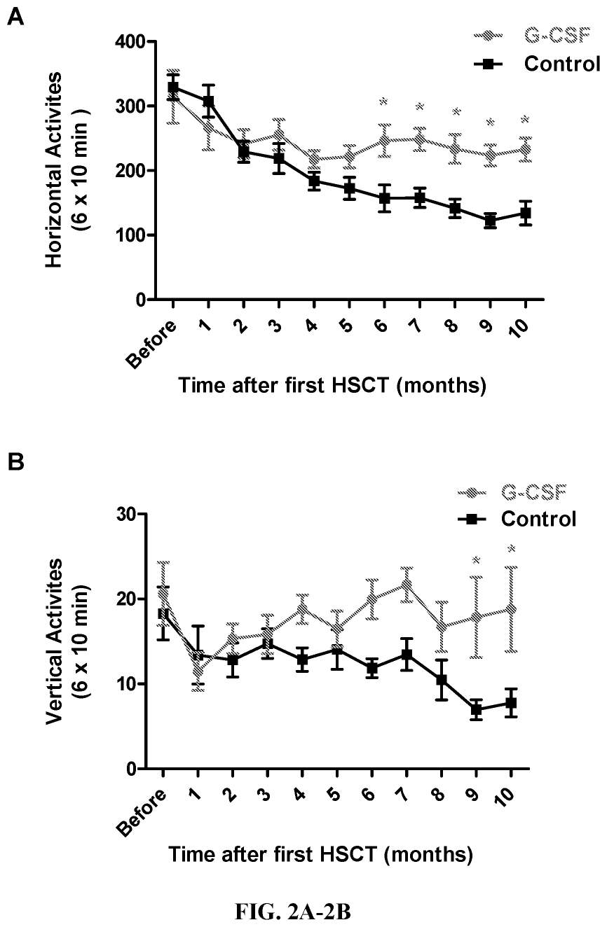

As shown in FIGS. 2A and 2B. The control mice without young HSCT displayed a decline in both horizontal and vertical locomotor activities with age. In contrast, these age-associated decline in motor function can be retarded by multiple young HSCT.

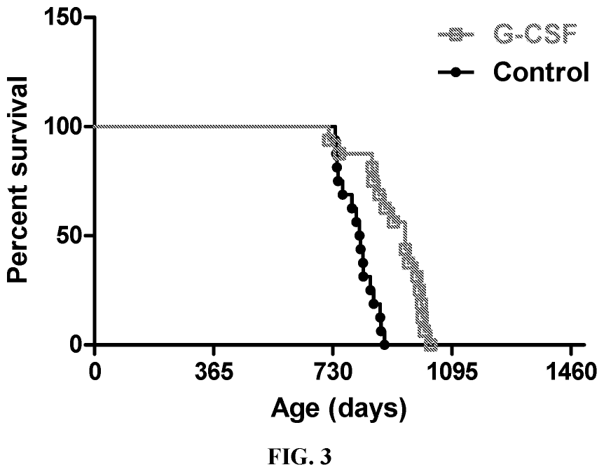

Effect of G-CSF aided young HSC transplantation on lifespan of aged GFP mice—The survival curves for the young HSC transplanted mice and control mice are presented in FIG. 3. The data were analyzed using GraphPad Prism 5.03 (GraphPad Software, La Jolla, Calif.). The homogeneity of survival curves between two groups were analyzed by log-rank test. The median life spans of the young HSC-transplanted mice was 952 days, which was 17% longer than that of control mice (813 days).

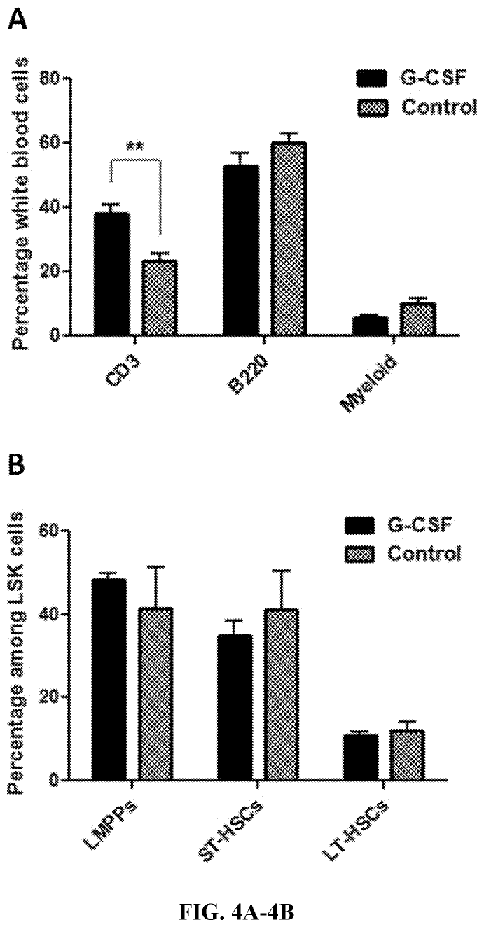

Effect of G-CSF aided young HSC transplantation on hematopoiesis in aged GFP mice—Peripheral blood (PB) and bone marrow (BM) cells were collected from 28-month old, 6 month after 8 times 10-week old HSCs transplanted GFP+ mice. The hematopoietic cell lineage were determined by cell immunostaining with anti-CD3ε, -B220, -CD11b and -Gr-1 antibodies for PB cells, and anti-Sca-1, -c-Kit, -CD34, -Flk-2, -streptavidin antibodies for BM cells, followed by FACS analysis, and data are presented in FIG. 4. Our results indicate that the blood cell rejuvenation by multiple G-CSF aided young HSC transplantation could extend healthspan (improvement in locomotor activities) and lifespan that may associate, in part, with the change in lineage of hematopoietic stem cells in aged mice.

Aging is the greatest risk factor for most common chronic diseases and as such is a major driver of health care expenditures. Thus, improving healthy aging has been progressively adopted as a wise and essential health care strategy. Healthy aging and longevity, a widely desired personal objective, can be potentially realized in our time thanks to major advances in science and technology, particularly biomedical research. The field of aging research has now moved into developing interventions that enhance healthspan and lifespan in experimental animals. However, these interventions have many unsolved caveats and limitations. Take the front-runner rapamycin as an example. This compound has been tested in mice and shown to extend mean and maximum lifespans, as well as healthspan, but a growing list of side effects raises question as to whether this drug will ultimately be beneficial in humankind. Clearly, while studies continue on these promising anti-aging mechanisms and interventions, new ideas and approaches are needed.

1. A method of stem cell rejuvenation comprising:
(a) administering at least one stem cell mobilization agent to a subject, wherein a target stem cell population migrates from host niches into the subject's blood;
(b) removing the target stem cells from the subject's blood;
(c) administering an effective amount of replacement stem cells to the subject, wherein the replacement stem cells are autologous stem cells previously isolated from the subject; and
(d) repeating steps (a)-(c) four or more times.

2. The method of claim 1, wherein the mobilized target stem cells are removed by apheresis.

3. The method of claim 2, wherein the mobilized target cells are collected and stored or conditioned.

4. The method of claim 1, wherein the replacement stems have been manipulated ex vivo.

5. The method of claim 4, wherein the replacement stem cells are conditioned autologous stem cells.

6. The method of claim 1, wherein the target stem cells are hematopoietic stem cells.

7. The method of claim 1, wherein the replacement stem cells are hematopoietic stem cells.

8. The method of claim 1, further comprising administering the mobilization agent prior to administering the replacement stem cells to the subject.

9. The method of claim 1, wherein a first mobilization agent is granulocyte-colony stimulating factor.

10. The method of claim 1, further comprising administering a second mobilization agent.

11. The method of claim 10, wherein the second mobilization agent is AMD3100.

12. The method of claim 1, wherein the replacement stem cells are genetically modified and/or in vitro conditioned stem cells.
